(19) United States
(12) Patent Application Publication
(10) Pub. No.: US 2008/0119686 A1
(43) Pub. Date: May 22, 2008

(54) Slotted plaque therapy device

(76) Inventor: Paul T. Finger, New York, NY (US)

Correspondence Address: WILLIAM B. GOWANLOCK, 16 TOWN CRIER LANE, MADISON, CT, 06443, US

(21) Appl. No.: 11/544,412

(22) Filed: Oct. 5, 2006

(51) Int. Cl. A61N 5/00

(52) U.S. Cl. 600/3

(57) ABSTRACT

A slotted plaque having a slot dimensioned (having a width and length) to permit the slotted plaque to be mounted on an organ's surface, such as an eye, in the area of a protrusion therefrom, such as the optic nerve sheath, to treat an intra-organ intraocular melanoma, such as an intraocular melanoma, having a contralateral component.

### FIELD OF THE INVENTION

The present invention relates to radiation therapy, and more specifically to a slotted plaque for applying radiation to an intra-organ cancer, such as an intraocular melanoma.

### BACKGROUND

Intra-organ melanomas are cancers that occur inside an organ. One type of intra-organ melanoma is an intraocular melanoma. As the name suggests, an intraocular melanoma is disease inside of an eye. More specifically, it may refer to cancer cells located in a part of the eye called the uvea. Some structures of the uvea in which intraocular melanomas are located include the iris, the ciliary body and the choroid. Intraocular melanomas may also be located on the optic disc. It should be appreciated that melanomas originating in one location of an organ may grow to involve another location of the organ. For example, an intraocular melanoma originating on the uvea might grow onto the optic disc.

Intraocular melanomas are classified into three general categories based on size. In accordance with the American Joint Committee on Cancer (AJCC) and COMS Staging Criteria, intraocular melanomas up to 2.4 millimeters in height and less than 10 mm in width are classified as T1 or small. Intraocular melanomas that are more than 2.4 to 9.9 millimeters in height and less than 16 mm in width are considered T2 or medium. Those intraocular melanomas that are 10 mm or larger in height and/or 16 mm or larger in width are T3 or COMS-large.

For some large intraocular melanomas, the only reasonable treatment option is enucleation (i.e., removal of the eye). While enucleation always remains an option, for other sizes of intraocular melanomas, another treatment known as plaque therapy may be appropriate, location permitting.

Plaque therapy offers the advantages of varying degrees of sight retention (after treatment). Additionally, the eye structure can be saved thereby eliminating the need for a cosmetic ocular prosthesis, such as an artificial eye. In addition, studies evaluating the effectiveness of plaque therapy verses enucleation (for medium-sized choroidal melanomas) have shown that plaque therapy is equally effective as enucleation in preventing metastatic disease and death. As a result, where appropriate plaque therapy is the standard of care.

Plaque therapy is a special form of radiation therapy. In the therapy, a plaque, which is a small generally metallic object containing radioisotopes (e.g., radioactive seeds), is surgically implanted on the exterior surface of the eye. More specifically, the plaque is sutured to the outside wall of the eye (i.e., the sclera) proximate the intraocular melanoma located therein. The radioisotope associated with the plaque emits radiation that penetrates the sclera. Once the radiation is within the eye, it encounters the intraocular melanoma. The plaque generally remains on the eye until the intraocular melanoma has received a therapeutic dosage of radiation (e.g., enough to destroy it). The plaque is then surgically removed.

Plaques are available in numerous shapes and sizes. These various shapes and sizes permit a surgeon to select a plaque most appropriate for treating a specific intraocular melanoma. In some cases, plaques may have a predetermined radiation distribution and dosage. In other cases, plaques may be customized by having a specific distribution of and type of radioisotope affixed thereto.

As indicated above, the structures within the eye generally susceptible to intraocular melanomas are the uvea (iris, ciliary body and choroid) and optic disc. The choroid comprises mainly blood vessels and is located between the sclera and the retina. It extends almost all around the eye, except it terminates at the ciliary body and optic disc.

The retina is connected to the eye's optic nerve at the optic disc. The optic nerve is located at the back of the eye (e.g., opposite the lens proximate the central fovea of the macula lutea). The optic nerve transits through the back of the eye by passing through the sclera. Where the optic nerve exits the eye, it is enclosed within an optic nerve sheath along with the eye's central retinal artery and central retinal vein.

Emanating from the uvea, intraocular melanomas can extend anywhere within the eye. Where the intraocular melanoma is some distance from the optic disc, a plaque can be selected and sutured to the sclera wherein the plaque is of an appropriate size such that the radioisotopes therewith provide a therapeutic dosage of radiation. Where an intraocular melanoma's location is proximate the optic disc, covers at least a portion of the optic disc, surrounds at least some portion of the optic disc, or some combination of the foregoing, use of a plaque becomes more problematic, or infeasible.

Similarly for intraocular melanomas emanating from the optic disc, whether entirely within the disc, or extending outwardly from the disc, use of a plaque can be problematic, or infeasible.

The use of a plaque becomes problematic or infeasible because its placement on the sclera proximate the intraocular melanoma is interfered with by the optic nerve sheath. More specifically, the plaque is prevented from being placed on the sclera in a position that permits the radioisotopes associated therewith to deliver a therapeutic dosage of radiation because of the contact of the plaque with the optic nerve sheath.

In some situations to overcome this problem, surgeons have shaved, or created shallow notches in, the plaque to remove the excess material abutting the optic nerve sheath to permit closer placement of the radioisotope to the optic nerve sheath. However, in most all cases of intraocular melanomas that are somehow associated with, or proximate, the optic disc, the optic nerve sheath simply prevents optimum, or acceptable, placement of the plaque, thus the radioisotope associated therewith. More specifically, the optic nerve sheath prevents the plaque from properly covering both the melanoma's ipsilateral and contralateral portions. As a result, intraocular melanomas that might otherwise have been effectively treated using plaque therapy cannot be which forces enucleation.

It is clear that plaque therapy offers patients suffering an intraocular, or intra-organ, melanoma a preferred treatment. What is needed in the art is a plaque that can be used on intra-organ melanomas, such as intraocular melanomas, where use of current plaques is problematic, or impossible, due to the intra-organ melanoma's location relative to a protrusion, such as the optic nerve sheath.

### SUMMARY OF THE INVENTION

This invention is a slotted plaque. The slot is dimensioned (having a width and length) to permit the slotted plaque to be mounted on an organ's surface, such as an eye, in the area of a protrusion therefrom, such as the optic nerve sheath, to treat an intra-organ intraocular melanoma, such as an intraocular melanoma, proximate the protrusion.

These and other features, aspects, and advantages of embodiments of the present invention will become apparent with reference to the following description in conjunction with the accompanying drawings. It is to be understood, however, that the drawings are designed solely for the purposes of illustration and not as a definition of the limits of the invention.

### BRIEF DESCRIPTION OF THE DRAWINGS

FIG. 1 is a cross-sectional view of the eye showing the structures of the eye relevant to the invention.

FIG. 2 is a series of views of retinal diagrams taken down the eye's visual axis showing intraocular melanomas located proximate the optic nerve and optic disc.

FIG. 3 is a side view, FIG. 3A, and an underside view (see line 3B in FIG. 3A), FIG. 3B, of a slotted plaque of the present invention.

FIG. 4 is an outside side view of an eye having the slotted plaque of the present invention placed thereon.

FIG. 5 is a retinal diagram showing the slotted plaque of FIG. 4 placed on the eye.

FIG. 6 is a series of views of a number of slotted plaques of the present invention that could be used to treat a given intra-organ melanoma around a given protrusion.

### DETAILED DESCRIPTION

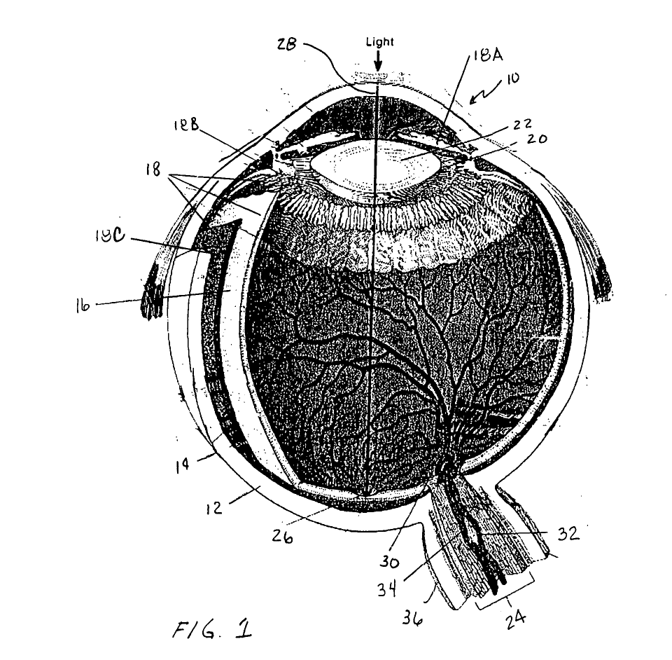

As shown in FIG. 1, an eye (generally referred to by the reference number 10) includes a sclera 12 that defines an outer surface 14 of the eye. Relevant structures for the present invention within the sclera 12 are a retina 16 and a uvea 18 (including the iris 18A, the ciliary body 18B and the choroid 18C), which is positioned between the retina and the sclera. The choroid 18C lines most of the sclera 12, but terminates at a suspensory ligament 20, which secures the eye's lens 22.

Extending from the retina 16 and out of the eye 10 is the optic nerve 24. The optic nerve 24 exits the eye 10 proximate the eye's central fovea of macula lutea 26 (or simply “fovea”), which is at the terminus of the eye's visual axis 28.

As the optic nerve 24 extends from inside of the eye 10, where it is attached to the retina 16 forming an optic disc 30, to the outside of the eye, it first passes through the choroid 18C and then the sclera 12. Where the optic nerve 24, as well as a central retinal artery 32 and a central retinal vein 34, exits the eye 10, it is contained within an optic nerve sheath 36, which is comprised of dura mater.

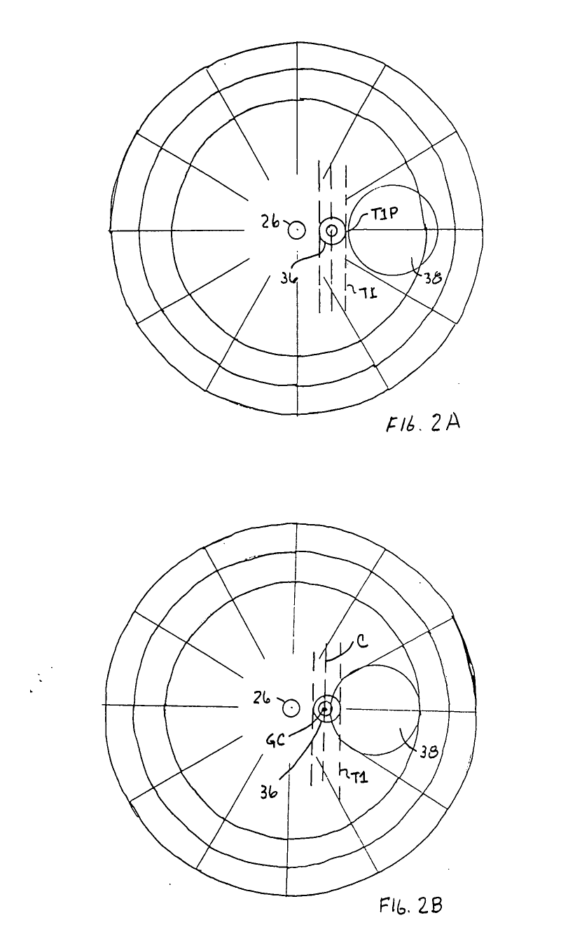

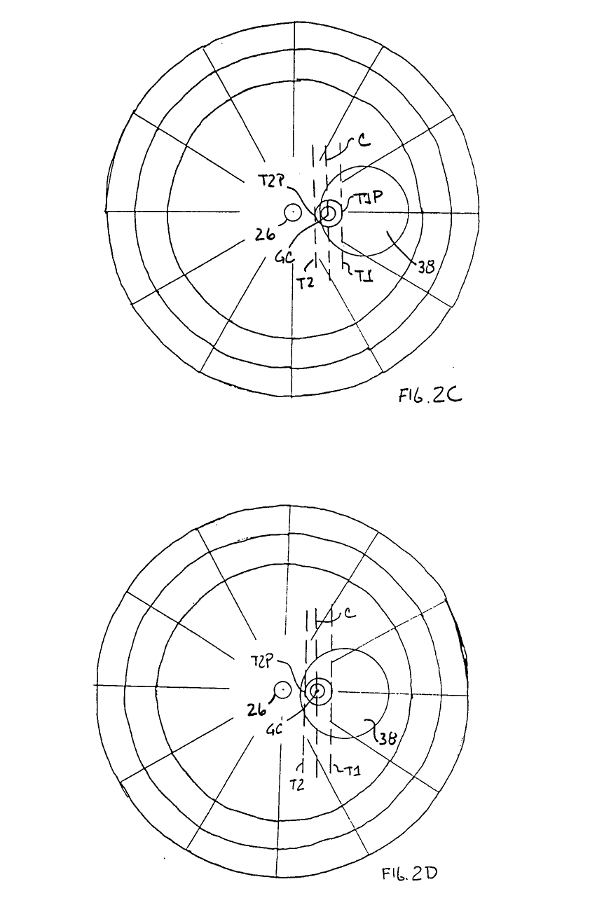

FIG. 2 is a series of retinal diagrams showing various placements of intraocular melanomas 38. Referring to FIG. 1, a retinal diagram is made from the perspective of looking through the lens 22 of the eye 10 down the visual axis 28. Thus, the center of the retinal diagram is the fovea 26.

Intraocular melanomas 38 can occur anywhere in the uvea 18, and on the optic disc 30, and be of almost any shape. In the area proximate the optic disc 30, the intraocular melanomas originating on the choroid 18C may grow around the optic disc, or, because of a short intraocular height of the optic disc, over at least some portion of the optic disc. In a rare case, an intraocular melanoma may originate on the optic disc 30 and then may grow onto the choroid 18C. Based on the foregoing, various intraocular melanoma placement types can be identified based upon the intraocular melanoma's location relative the optic nerve sheath 36.

FIG. 2A shows an intraocular melanoma 38 of a first placement type. A first placement type intraocular melanoma 38 exists when the intraocular melanoma is proximate the optic nerve sheath 36, but there is no tangent T1, which has a tangent point T1P, to the optic nerve sheath that can be drawn such that the tangent is in contact with the intraocular melanoma on both sides of the tangent point.

Continuing with FIG. 2B, FIG. 2B shows an intraocular melanoma 38 of a second placement type. A second placement type intraocular melanoma 38 exists when there is a tangent T1, which has a tangent point T1P, that can be drawn such that the tangent contacts the intraocular melanoma on both sides of the tangent point. However, there is generally no centerline C, which is through the optic nerve sheath's geometric center GC, such that the centerline contacts the intraocular melanoma on both sides of the centerline. In the rare instance where it does, all of the intraocular melanoma is on only one side of the centerline.

A third placement type of intraocular melanoma 38 is shown in FIG. 2C. As shown for this third placement type, the intraocular melanoma 38 meets the tangent T1 criteria of the second placement type. However, a centerline C can be drawn that contacts the intraocular melanoma on both sides of the geometric center GC and results in some of the intraocular melanoma being on both sides of the centerline. Additionally, there is another tangent T2 to the optic nerve sheath 36, having another tangent point T2P, that can be drawn, that does not contact the intraocular melanoma on both sides of the other tangent point.

FIG. 2D shows a fourth placement type of an intraocular melanoma 38. This fourth placement type meets the tangent T1 requirement, and centerline C requirement of the third placement type, but there is no other tangent T2 that can be drawn that does not contact the intraocular melanoma 38 on both sides of the other tangent's tangent point T2P.

There is a rare case (not shown) where the intraocular melanoma 38 is entirely within the area defined by the optic nerve sheath 36, be it on the choroid 18C, on the optic disc 30, or some combination. In this case, where a centerline C through the geometric center of the optic nerve sheath 36 can be drawn that does not contact the intraocular melanoma 38, this is a fifth placement type. If this is not possible, this is a sixth placement type.

It should be appreciated that intraocular melanomas 38 of the first, second and fifth placement types have all of the intraocular melanoma on one the side of a centerline C. While, intraocular melanomas 38 of the third, fourth and sixth placement types have at least some portion of the intraocular Melanoma on both sides the centerline C.

In summary, intra-organ melanomas having placement types of the first, second, and fifth type have only an ipsilateral component (i.e., all the intra-organ melanoma is on the same side of a centerline). While placement types of the third, fourth, and sixth types have both an ipsilateral and a contralateral component. Where there is both an ipsilateral and contralateral component, it should also be appreciated that the ipsilateral component is not necessarily coincidental with the largest mass of the intra-organ melanoma just as the contralateral component is not necessarily coincidental with the smallest mass.

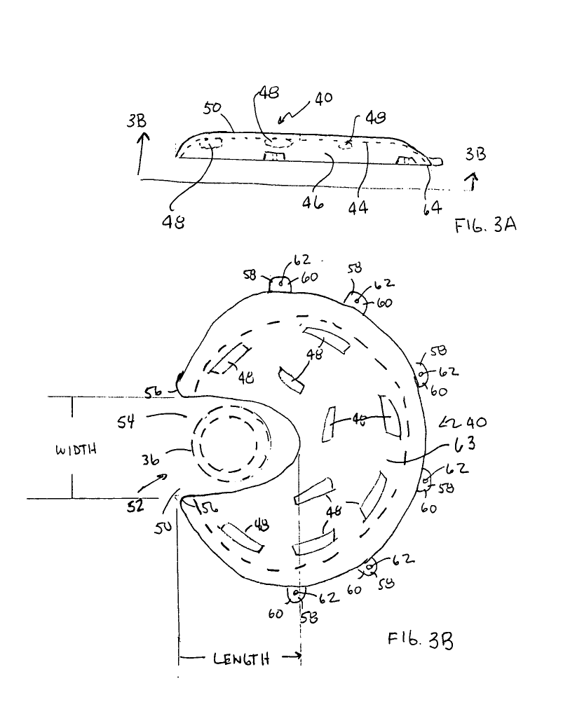

As shown in FIGS. 3A and 3B, a slotted plaque of the present invention (generally referred to by reference number 40) includes a body 42. The illustrative body 42 has an inner surface 44, which is concave, that defines a volume 46. The body 42 has radioisotopes 48 associated therewith, which are discussed in detail below.

The body 42 further defines a slot 50. The slot 50 has an opening 52 dimensioned to permit the optic nerve sheath 36 (shown in dotted lines) to enter the body 42. Further, the slot 50 has a passage 54 of sufficient length and width to allow at least some portion of the optic nerve sheath 36 to travel some depth into the body 42.

It should be appreciated that the dimensions of the slot 50 are based in large part on the diameter of the optic nerve sheath's 36 cross-section in the area proximate the eye 10 (see FIG. 1). It is anticipated that a slotted plaque 40 of the present invention will be available in several sizes with various slot 50 sizes to accommodate variations in the size of optic nerve sheaths 36 and intraocular melanomas 38.

That said, typically the cross-section of optic nerve sheath 36 has a diameter of 4 to 5 mm. As the working room behind the eye 10 for implantation of a slotted plaque 40 is quite small, a practical minimum slot opening 52 is 4 mm larger than the optic nerve sheath 36. Thus, an average slot 50 should have an opening 52 of about 8 mm. The passage 54 down the length of the slot 50 should also be about 8 mm wide, but it could vary. It should be appreciated that the body 42 may define a slot 50 with chamfers 56 that permit easier insertion of the optic nerve sheath 36 therein. The length of the slot 50 will be discussed in detail below.

The body 42 may also incorporate attachment points 58. Attachment points 58 permit the slotted plaque 40 to be affixed to an eye 10. The specific attachment points 58 illustrated are for use with sutures. The attachment point 58 includes an appendage 60 extending outwardly from the body 42 and defining a hole 62. In use, a suture would be placed through the hole 62. Other attachment points 58 might simply provide a surface sufficient to accept an adhesive to provide a proper bonding strength.

The body 42 is most appropriately made of a material that provides radiation shielding. More specifically, as discussed above, the body 42 has radioisotopes 48 associated therewith, such as in the form of seeds. In use, the body 42 is placed on an organ, such as the eye, with the objective of exposing an intra-organ melanoma, such as an intraocular melanoma, to the radiation emanating from the body. Radiation from a point source is emitted in all directions unless shielding is provided. A body 42 made of, or incorporating, a material having a shielding capability, such as gold, will prevent other organs from being exposed to significant amounts of inadvertent radiation. In some cases, inadvertent radiation exposure can be reduced by up to 99%.

As discussed above, slotted plaques 40 have radioisotopes 48 associate therewith. As illustrated, the radioisotopes are in the form of seeds placed on the interior surface 44 of the body 42. Radioisotopes 48 in seed form currently include palladium-103, iodine-125 and cesium-131. Use of seeds allows a surgeon to design both the dosage and dosage pattern. Other slotted plaques may have a radioisotope, such as ruthenium-106, integrated therein.

The body 42 is of a size relative to the intraocular melanoma 38 such that the seeds can be placed thereon in a pattern to have a therapeutic dosage of radiation reach the entire intraocular melanoma. While the precise placement of individual seeds will be discussed below, the radioisotopes 48 define a therapeutic portion 63. A slotted plaque 40 is selected such that a therapeutic portion 63 will extend 2 mm beyond an intra-organ's margin.

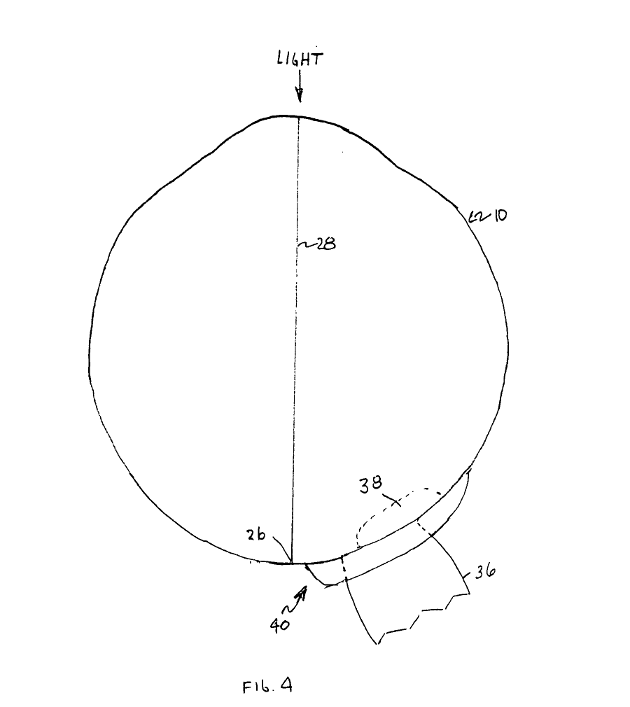

Referring to FIG. 4, the slotted plaque 40 is attached to eye 10 in an area proximate the optic nerve sheath 36 to treat an intraocular melanoma 38.

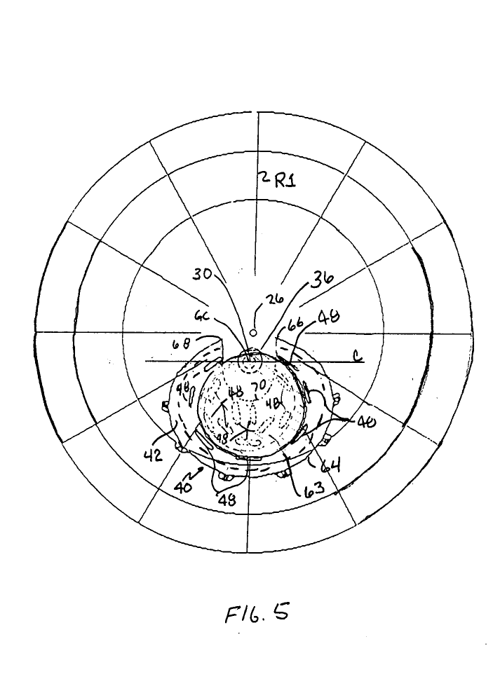

Continuing with FIG. 5, the placement of the slotted plaque 40 relative to an intraocular melanoma 38 can be seen. FIG. 5 is a retinal diagram where the view is taken down the visual axis (see FIG. 1). As a result, the intraocular melanoma 38 is on top of the slotted plaque 40 (which is oriented bottom to top). Additionally, the optic disc 30 and the optic nerve sheath 36 are located between the intraocular melanoma 38 and the slotted plaque 40 with the optic disc on top of the optic nerve sheath. Additionally, the sclera 12 has been removed for clarity.

In this case, the intraocular melanoma 38 of the third placement type is depicted. Referring to FIG. 2C, it is a third placement type because there is a tangent T1 to the optic nerve sheath 36 that can be drawn that contacts the intraocular melanoma 38 on each side of the tangent's point T1P. Additionally, there is a centerline C, through the geometric center GC of the optic nerve sheath 36, that can be drawn that contacts the intraocular melanoma 38 on each side of the geometric center defining ipsilateral and contralateral sides. There is, also, a tangent T2, to the optic nerve sheath 38, that can be drawn that does not contact the intraocular melanoma.

As shown, the slotted plaque 40, which is generally symmetrical about a radial R1, is placed over the intraocular melanoma 38. More specifically, the slotted plaque 40 has a first perimeter 64, which begins at a first location 66 on the slot 50 and extends to another location 68 on the slot. The slot 50 has a second perimeter 70 that continues from the other location 68 back to the first location 66.

The first perimeter 64 generally encircles the intraocular melanoma 38. The second perimeter 70, which defines the width and depth of the slot 50, permits sufficient width to allow the optic nerve sheath 36 to enter and travel down the slot. As depicted, the placement of the slotted plaque 40 places a majority of the slotted plaque 40 and a majority of the intraocular melanoma 38 on the one side of the centerline C, which is denoted the ipsilateral side based on how the plaque 40 is being applied. A minority portion of the slotted plaque 40 and the intraocular melanoma 38 are on the contralateral side of the centerline C. If this were reversed, the slot 50 might nearly bisect the body 42.

As shown in FIG. 5, a first grouping of radioisotope 48, which is in the form of seeds, is placed just outside and around the intraocular melanoma's margin 72. As discussed above, this first grouping of seeds 48 generally defines the therapeutic portion 63 of the slotted plaque 40. It only generally defines the therapeutic portion 63 because radiation from a point source extends outwardly in all directions, therefore the actual therapeutic portion may be slightly larger. Additionally, the therapeutic portion 63 may be irregular if seeds of different strengths are used. A second grouping of radioisotopes 48, which is also in the form of seeds, is placed within an area defined by the intraocular melanoma's margin 72. Ideally, there should be a 2 mm apparent free-margin of safety.

It should be appreciated that the slot 50 has sufficient length to permit radioisotope 48 placement outside the margin 72 on the contralateral component of the intraocular melanoma 38. It should also be appreciated that if the intraocular melanoma 38 extended further on the contralateral side (e.g., the fourth placement type), the slot 50 could have sufficient length to permit radioisotopes 48 to be placed within the margin 72 of that contralateral component of the intraocular melanoma 38.

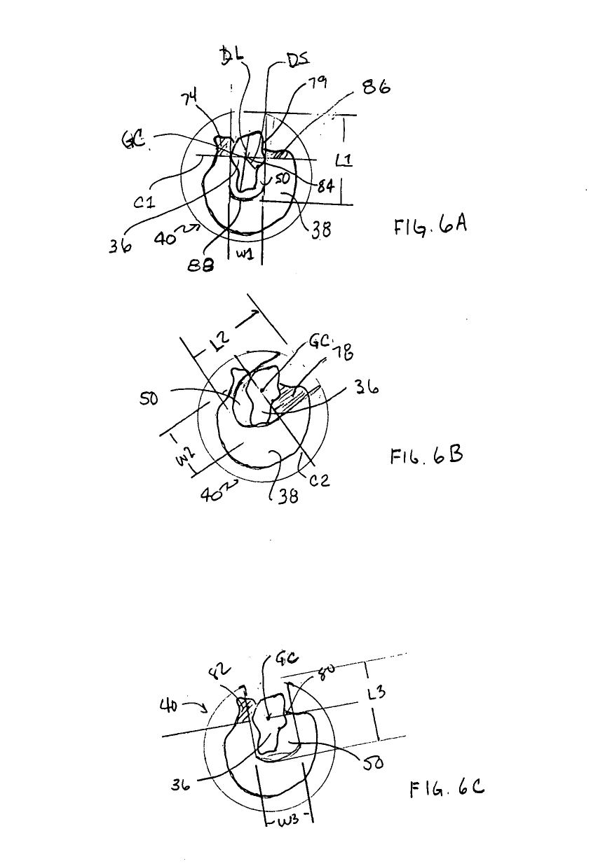

As shown in FIG. 6, a single intraocular melanoma 38 may be treated by one of several slotted plaques 40. While the slotted plaque 40 selected for treatment is ideally the one that will provide the most effective treatment, circumstances may dictate a less than optimum selection. Therefore, it is important to note the different treatment options and the differences in structural characteristics of the slotted plaque 40 they may dictate.

As illustrated in FIG. 6, the intraocular melanoma 38 is of the third placement type. The protrusion 36 has an irregular shape such that the geometric center GC is not symmetrically located.

As shown in FIG. 6A, a centerline C1 through the geometric center GC of the protrusion 36 can be drawn that establishes two contralateral components 74, 76 of the intra-organ melanoma 38. The slotted plaque 40 is symmetrical with a slot 50 of a length L1 and a width W1.

As shown in FIG. 6B, a centerline C2 through the geometric center GC of the protrusion 36 can been drawn that establishes one contralateral component 78. As a result, the slot 50 is offset, thus the slotted plaque 40 is asymmetrical. The slot 50 has a length L2 and a width W2. In this case as FIG. 6A and 6B are generally to the same scale, L2 is approximately equal to L1, but W2 is greater than W1.

As shown in FIG. 6C, a centerline C3 through the geometric center GC of the protrusion 36 can be drawn that establishes two different contralateral components 80, 82. As a result, the slot 50 is offset, thus the slotted plaque 40 is asymmetrical. The slot 50 has a length of L3 and a width W3. In this case as FIG. 6A, 6B and 6C are generally to the same scale, L3 is shorter than both L1 and L2, but W3 is greater than W1 and less than W2.

The above cases, while not comprehensive, show some of the possible variations in slotted plaque 40 designs. In all cases, however, the slot 50 must permit the positioning of the geometric center GC of the protrusion 36 at some depth within the slotted plaque 40. It should be appreciated that the slot 50 defines a discontinuity in the slotted plaque 40, thus in the area of the slot there is no dosage of radiation. The surgeon must therefore assess the radiation field as to the no dosage area of the slotted plaque 40 to assure optimum treatment.

Referring to FIG. 6A, the protrusion has a shortest distance DS, which is from the geometric center GC to a point 84 which is the nearest point on the protrusion's 36 perimeter 86, and a longest distance DL, which is from the geometric center CG to a another point 88, which is the farthest point on the protrusion's perimeter 86. Where the slot 50 is relatively straight, the slot should have a length greater than the shortest distance DS. Thus, the slot 50 should be dimensioned to accept the protrusion 36 at least to a depth equal to the shortest distance DS. In some cases, the slot 50 should be dimensioned to accept the protrusion 36 at least to a depth equal to the longest distance DL. Where the majority of the mass on the intra-organ melanoma 38 is on the contralateral side, the slot 50 might nearly bisect the body. In the case where the shortest distance DS equals the longest distance DL, the protrusion has a circular cross-section.

It should also be noted that in all these examples, the slotted plaque 40 is placed such that the intra-organ melanoma 38 is within the perimeter defined by the slotted plaque 40. Ideally, the maximum amount of intra-organ melanoma 38 is within the therapeutic portion 63 of the slotted plaque 40. In some cases, this may not be the case.

The above slotted plaque 40 is for use in plaque therapy. More specifically, the slotted plaque is for plaque therapy on an organ having an intra-organ melanoma, where the organ has a protrusion that interferes with placement of a plaque. Additionally, the intra-organ melanoma has a contralateral component.

In use, a surgeon would identify an organ having an intra-organ melanoma. The surgeon would also identify that the intra-organ melanoma is involved with a protrusion from the organ such that the intra-organ melanoma has a contralateral component.

The surgeon would also evaluate whether the intra-organ melanoma is a candidate for plaque therapy based on the type and size of the melanoma.

If the intra-organ is a candidate for plaque therapy, a slotted plaque would be obtained. The slotted plaque would have a therapeutic area and a slot. The slot would be dimensioned such that at least a portion of the therapeutic portion could treat the contralateral component of the intra-organ melanoma.

The surgeon would then attach the slotted plaque to the organ. After attachment, at least a portion of the protrusion would be within the plaque.

After attachment, the slotted plaque would be left in place until a therapeutic dosage of radiation is applied, or it is determined that it should be removed for other reasons, thus delivering at least some portion of a therapeutic dosage. The slotted plaque would then be removed.

While the steps have been presented in an order, the order should be considered arbitrary where a particular step need not absolutely follow or come before other steps presented before or after it.

While there has been illustrated and described what is at present considered to be preferred and alternative embodiments of the claimed invention, it will be appreciated that numerous changes and modifications are likely to occur to those skilled in the art. It should also be appreciated that while the invention has been shown for use with an eye, other organs of similar structure (e.g., organs having a protrusion therefrom) on which brachytherapy could be used are considered within the scope of the invention. In the case of other organs, the slot should be sized to accommodate the protrusion as discussed above for the optic nerve sheath. In addition, the term protrusion may include structures, such as muscle and tendons, that are attached to an organ. Additionally while the slotted plaque has been illustrated as round, other shapes are possible, thus this is not a requirement of the invention. Additionally, while the slot has been shown as relatively straight, this is not a requirement of the invention. The issue is the final placement of the protrusion within the slotted plaque such that the slotted plaque can provide treatment to a contralateral side of an intra-organ melanoma. It is intended in the appended claims to cover all those changes and modifications that fall within the spirit and scope of the claimed invention.

What is claimed is:

1. A slotted plaque for use in plaque therapy on an organ having a protrusion and an intra-organ melanoma, where the intra-organ melanoma has a contralateral component, the slotted plaque comprising:
a body, the body defining a slot;
radioisotopes associated with the body defining a therapeutic portion;
wherein the slot is dimensioned to accept a protrusion from an organ where the organ has an intra-organ melanoma having a contralateral side, such that the therapeutic portion extends to the contralateral side.

2. The slotted plaque of claim 1 wherein the organ is an eye having an optic nerve sheath, and the protrusion is the optic nerve sheath.

3. The slotted plaque of claim 1 wherein the radioisotopes include radioactive seeds.

4. The slotted plaque of claim 1 wherein the slot has chamfers.

5. The slotted plaque of claim 1 wherein the body is symmetrical.

6. The slotted plaque of claim 1 wherein the body is asymmetrical.

7. The slotted plaque of claim 1 wherein the protrusion has a geometric center and a perimeter, and there is a shortest distance from the geometric center to the perimeter, and the slot permits the protrusion to enter the body to a depth at least equal to the shortest distance.

8. The slotted plaque of claim 1 wherein the protrusion has a geometric center and a perimeter, and there is a longest distance from the geometric center to the perimeter, and the slot permits the protrusion to enter the body to a depth at least equal to the longest distance.

9. A plaque therapy method for an organ having an intra-organ melanoma, where the organ has a protrusion and the intra-organ melanoma has a contralateral component, the method comprising the steps of:
identifying an organ having a protrusion and an intra-organ melanoma;
determining whether the intra-organ melanoma has a contralateral component due to its placement relative to the protrusion;
determining that the intra-organ melanoma is suitable for plaque therapy;
obtaining a slotted plaque having an amount of a radioisotope associated therewith to administer a therapeutic dosage to the intra-organ melanoma and a slot, the slot dimensioned to accept the protrusion such that at least a portion of the therapeutic portion aligns with at least a portion of the contralateral component;
attaching the slotted plaque to the organ such that the at least a portion of the therapeutic portion aligns with the at least a portion of the contralateral component; and
administering at least some portion of a therapeutic dosage; and
removing the slotted plaque after the at least some portion of a therapeutic dosage has been administered.

10. The method of claim 9 wherein in the step of administering at least some portion of a therapeutic dosage, the dosage is complete.

11. The method of claim 9 wherein in the step of identifying an organ, the organ is an eye, the protrusion is the optic nerve sheath, and the intra-organ melanoma is an intraocular melanoma.

12. The method of claim 9 wherein in the step of obtaining a slotted plaque, the slotted plaque has a body that is symmetrical.

13. The method of claim 9 wherein in the step of obtaining a slotted plaque, the slotted plaque has a body that is asymmetrical.
